(19) United States
(12) Patent Application Publication
(10) Pub. No.: US 2009/0283494 A1
(43) Pub. Date: Nov. 19, 2009

(54) EASY CLEAN REFILLABLE BOTTLE AND DISPENSING CAP

(75) Inventor: Charles P. Maxwell, Mequon, WI (US)

Correspondence Address: PEARNE & GORDON LLP, 1801 EAST 9TH STREET, SUITE 1200, CLEVELAND, OH, 44114-3108, US

(73) Assignee: Tablecraft Products Company, Gurnee, IL (US)

(21) Appl. No.: 12/120,283

(22) Filed: May 14, 2008

(51) Int. Cl. B65D 41/34; B65B 7/28; A61L 2/02; B65D 37/00; B65D 3/04

(52) U.S. Cl. 215/329; 215/44; 215/382; 422/25; 222/206

(57) ABSTRACT

A plastic refillable bottle and a plastic screw-on closure for the bottle, the bottle having a one-piece molded hollow body and integral neck, the walls of the body and neck being of generally uniform thickness, the neck having an opening through which material in the bottle passes when being dispensed and being formed with a helical thread, the closure being configured to extend across the bottle mouth and having a skirt for overlying the bottle neck, the closure skirt having a thread complementary to the thread on the neck and enabling the closure to be tightened onto the neck and effect a seal adequate to avoid excessive leakage of the contents out of the bottle through an interface between the skirt and neck, interior surfaces of the bottle and closure including the areas of the bottle and closure forming the respective threads being devoid of concave surface areas with a radius smaller than about ⅛″ at corners forming an angle of less than 135°.

### BACKGROUND OF THE INVENTION

The invention relates to food containers and, more particularly, to refillable containers and closures threaded onto the containers.

### PRIOR ART

In restaurants and other food service establishments, food containers of a size convenient to be hand-held are frequently refilled from larger containers or from a spigot, for example. This practice can reduce cost since goods originally packaged and shipped in large bulk containers can be considerably less expensive than goods filled, shipped and distributed in relatively smaller packages.

It is recognized that food containers should be sanitized by washing them clean from time-to-time and, specifically, prior to each refilling. With prior art containers, it can be difficult to effectively and reliably clean the interior spaces of the container and its companion closure.

Prior art containers in the form of bottles or jars with screw-on closures, i.e. tops or caps, can present areas that are difficult to clean by hand and/or by machine. The screw threads and frequently other areas of the container and closure present tight inside corners, i.e. concave areas with small radii of curvature that physically trap food particles making them difficult to completely remove. Container and closure designers have largely followed a practice of proportioning the thread forms with dimensions comparable to the thickness of the container or closure walls. This constraint necessarily results in sharp corners in order for a thread to have a traditional profile or form. The angular thread profile typically found in threaded closures presumably is specified by designers to ensure that a reliable and sufficient axial thrust will be developed between the thread elements so that a seal will be established at the mouth of the container when the closure is torqued onto the container but that the threads won't strip or slip under the required torque.

Tight inside corners, i.e. less than 135° tend to retain food particles in part because scrubbing and/or flushing action of cleaning cloths, brushes and water streams or flooding and the like are obstructed by the adjacent surfaces of the container or closure. When residual food particles remain on or in the container or closure, a risk of bacteria growth exists that can lead to food poisoning. NSF International (NSFI) has recommended radii of at least ⅛″ at inside corners for food equipment and devices where small radii can be avoided but allows for smaller radii where necessary for proper functions of parts.

### SUMMARY OF THE INVENTION

The invention provides a refillable bottle or like container and a dispensing closure or top especially suited for use with food products by virtue of being relatively easy to sanitize. The bottle and top are both characterized by smooth profiles devoid of sharp inside corners thereby avoiding areas that are prone to accumulate and retain food particles when washed, scrubbed, rinsed, and otherwise subjected to sanitizing procedures. Smooth contours of the bottle and closure components, in accordance with the invention, are maintained throughout their interiors including areas at the bottle mouth and the juncture of the closure end wall and skirt and even those areas formed with mating threads.

The invention is especially suited for thin wall plastic bottles and like containers and particularly, bottles that are semi-rigid and can be squeezed to forcibly express their contents. The disclosed bottle can be used, when suitably configured and made of appropriate material such as low density polyethylene, to squeeze dispense a wide variety of foods as well as non-food materials. Examples of edible materials in liquid, paste, and dry particulate flowable form include salad dressings, vegetable oil, water, sauces, ketchup and mustard, and other condiments, dried vegetables, spices, seeds, cereals, crumbs, and so forth.

In accordance with the invention, the container bottle and closure top have unique complementarily shaped threads enabling the top to be screwed onto the bottle and a gasketless liquid tight seal to be easily and reliably established. The respective walls of the bottle and top are formed into threads that have rounded forms or profiles. The radial root to crest dimension of the threads is, according to the invention, large in comparison to the respective wall thickness.

In addition to providing surface contours that are easy to clean, the disclosed thread construction of the bottle and top operates in a novel manner to improve the ease and the reliability of a gasketless seal between these elements. More particularly, the wall areas of the bottle and top that provide the mating threads are configured in a manner analogous to an accordion. As a result, they can compress or even elongate to accommodate small dimensional variations in these parts and still achieve a good seal without having to be excessively tightened. It can be difficult to produce a bottle and top combination with a consistent seal performance where the bottle is a squeeze dispensing type and the mouth of the bottle tends to deform when the bottle is squeezed. The thread forms of the invention in these circumstances are helpful in maintaining an adequate seal.

### BRIEF DESCRIPTION OF THE DRAWINGS

FIG. 1 is a perspective view of a bottle and top constructed in accordance with the invention;

FIG. 2 is a fragmentary side view, partially in cross-section of the bottle shown in FIG. 1;

FIG. 3 is a side view, partially in cross-section, of the top shown in FIG. 1; and

FIG. 4 is a fragmentary cross-sectional view of the top sealed on the bottle.

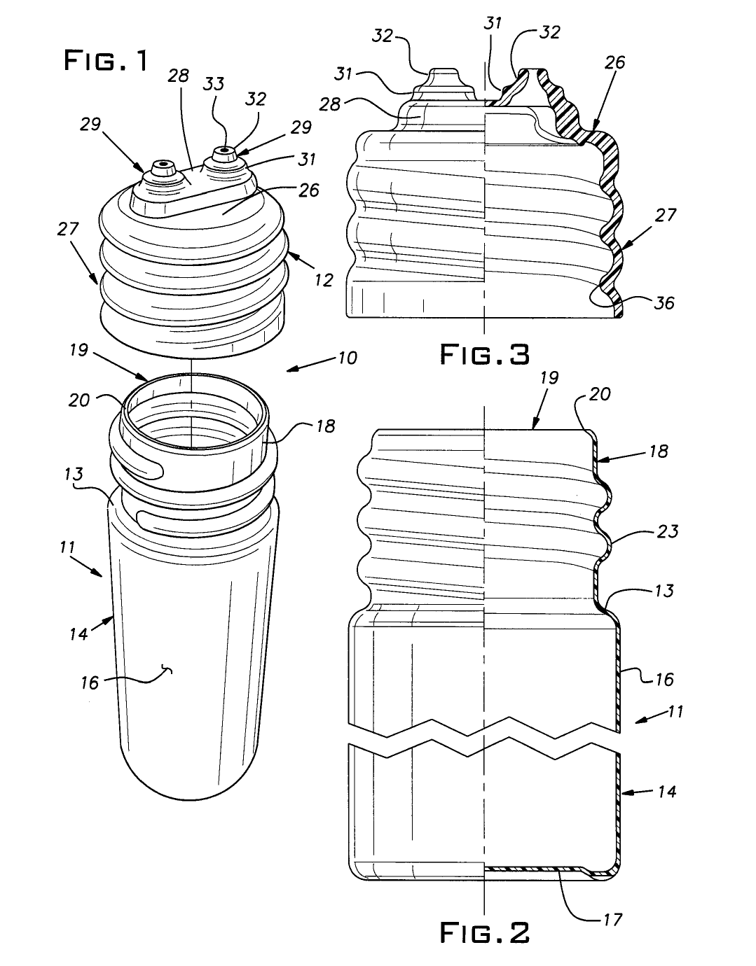

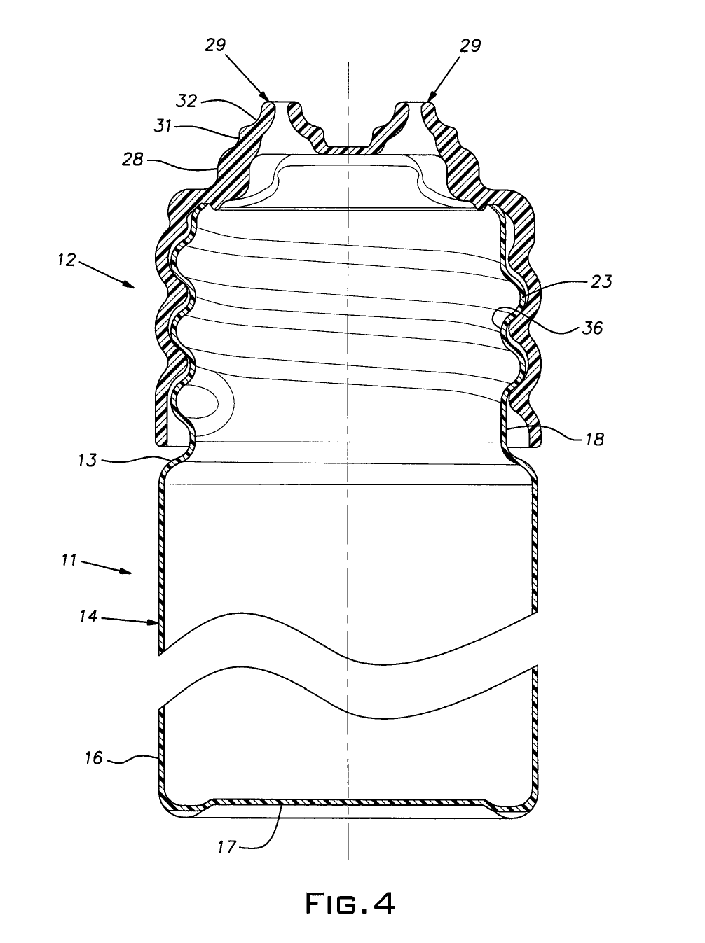

### DESCRIPTION OF THE PREFERRED EMBODIMENT

The figures illustrate a refillable container 10 comprising a bottle 11 and screw-on dispensing closure in the form of a cap or top 12 useful for containing and dispensing flowable materials in the form of liquids, pastes and particulate solids. It will be understood that the bottle 11 and top 12 are especially useful for containing and dispensing edible materials intended for human consumption.

The illustrated bottle 11 is preferably blow-molded and can be made of low density polyethylene or other suitable food grade thermoplastic material. The bottle 11, which in the illustrated example, is a nominal 20 ounce container, has a main body formed by a generally cylindrical sidewall 16, of about 2¾″ diameter, extending from a bottom or end wall 17 upwardly to a shoulder 13. The main body 14 of the bottle below the shoulder 13 has a length that is substantially greater than its transverse width or diameter. The diameter and length of the bottle make the bottle convenient to grasp and squeeze with the hand of an average size adult. A circular neck 18 of the bottle 11 concentric to the axis of the sidewall 16 has an open mouth 19 at its upper end. A circular lip 20 of the mouth 19 is in-turned from the neck 18 so that the lip has a substantial radial component to its orientation so that a liquid tight seal with the top end wall 26 is assured when the top is tightened. In the illustrated form of the bottle 11, the neck 18 and mouth 19 are relatively large or wide being of a diameter nearly the same as the diameter of the main body 14. It will be seen that the inside corner 21 formed between the bottom end wall 17 and sidewall 16 has a radius of at least about ⅛″. It will be understood that an inside corner surface will be an area where surfaces converge at an angle of 135° or less and the surface area under consideration is concave. It will, likewise, be understood that for the illustrated bottle 11, a radius dimension of ⅛″ is relatively large compared, for example, to a nominal generally uniform wall thickness of the bottle which, in the illustrated case is about 1 mm. The neck 18 is molded with an external screw thread 23. The illustrated thread 23 is a continuous helix extending through approximately two turns. The thread 23 has a profile that is rounded at both its crest and root. The crest-to-root radial dimension of the thread 23 is preferably considerably larger than the wall thickness of the bottle neck 18, being about at least 3 times larger. The profile or form of the external thread 23, owing to a generally uniform wall thickness in the bottle, is reflected directly in the corresponding interior surface of the neck. Note that where the external thread 23 is convex, the corresponding area on the interior of the neck 18 will be concave and, vice versa, where the thread is concave, the inside surface of the neck will be convex. In accordance with the invention, the thread 23 is proportioned such that all of the associated concave interior neck surfaces, i.e. those reflecting the contours of the convex parts of the external threads 23 have a relatively large radius preferably at least ⅛″ radius.

The top 12 is preferably an injection molded one-piece article formed of high density polyethylene or other suitable food grade relatively rigid plastic material. The top 12 includes an end wall 26 and a depending peripheral circular skirt 27. The top 12 is formed with a nominal generally uniform wall thickness of, for example, about 1.8 mm to about 2.0 mm. The end wall 26 has a diametral oblong raised area 28. At least one dispensing zone 29 (two are illustrated) in the form of a small hollow spout is molded atop the area 28. The spout 29 can be formed with rounded steps 31, 32 that create a narrowing cross-sectional discharge area. The user of the bottle and top container 10 can cut off one or more of the rounded steps 31, 32 or the raised area 28 to obtain a larger dispensing hole area or outlet. The distal tip of the outer step 31 can be originally molded with a small outlet hole 33. Preferably where a plurality of discharge spouts 29 are provided, they are arranged along a line diametral to the circular shape of the end wall 26 and skirt 27.

The skirt 27 of the top has an internal screw thread 36 configured to mate with the external thread 23 of the bottle 11. The thread 36 has a rounded profile or form with any inside corner concave surfaces on the inside of the skirt 27 preferably having a radius of at least ⅛″. Similarly, all of the remaining interior inside corner concave surface areas of the top 12 have radii of at least ⅛″ such as those existing at the steps 31, 32 of the spouts 29. It will be seen that the radial crest to root distance of the thread 36 is at least as large as the nominal wall thickness of the top 12. The exterior of the top skirt 27 has a corrugated profile reflecting the form of the internal thread 36. Ideally, external concave surface areas of the top have radii of at least ⅛″.

The disclosed thread form on the bottle 11 in conjunction with the large radius inside corners of the bottle as well as the surfaces of the top 12 that come into contact with the contents of the bottle once the top seals the bottle mouth 19 are readily scrubbed and washed for ease of sanitation. This is also the case with the external surfaces of the bottle and top again improving the cleanability and ultimately the sanitation of the container 10.

The disclosed thread forms of the bottle neck 18 and top skirt 27 give these parts the ability to radially axially compress and/or extend in a manner analogous to that of an accordion or corrugated hose. This feature enables the bottle mouth 19 to easily and reliably seal against the top end wall 26 to avoid excessive leakage through the interface of the skirt 27 and neck 18 without requiring excessive closing torque to be applied to the top when screwing it onto the bottle 11. This improved sealing capacity is an additional feature to that of the ready cleanability and improved sanitation results achieved by the relatively large radius of the mating thread forms of the bottle and top. Still further, the rounded thread forms serve to make the top and bottle self-aligning and smooth acting when tightened and minimize the risk of cross-threading when initially joined.

While the illustrated threads 23, 36 on the bottle 11 and top 12 are shown as extending continuously through two turns, it will be understood that a thread of greater or lesser turns and even an interrupted thread can be used on either or both the bottle and top although care should be taken to provide a rounded profile or form in the cross-section of any thread and at the start and end of any thread segment. The disclosed bottle, by virtue of its construction of relatively soft resilient material such as low density polyethylene and its length as compared to its transverse dimension is conveniently used as a squeeze bottle so as to forcibly dispense the contents of the bottle through the dispensing ports 29 in the top 12. The top 11 which is relatively rigid by virtue of its wall thickness and material composition serves to maintain the shape of the bottle neck when the bottle is squeezed and thereby assures that a seal will be maintained between the bottle mouth 19 and the top end wall 17 even in the absence of a gasket therebetween. Certain benefits of the invention can be obtained with the disclosed thread form when incorporated on large mouth bottles, often called jars, and on rigid or semi-rigid bottles or jars whether they be blow-molded, injection blow-molded, or entirely injection molded.

It should be evident that this disclosure is by way of example and that various changes may be made by adding, modifying or eliminating details without departing from the fair scope of the teaching contained in this disclosure. The invention is therefore not limited to particular details of this disclosure except to the extent that the following claims are necessarily so limited.

What is claimed is:

1. In combination, a plastic refillable bottle and a plastic screw-on closure for the bottle, the bottle having a one-piece molded hollow body and integral neck, the walls of the body and neck being of generally uniform thickness, the neck having an opening through which material in the bottle passes when being dispensed and being formed with a helical thread, the closure being configured to extend across the bottle mouth and having a skirt for overlying the bottle neck, the closure skirt having a thread complementary to the thread on the neck and enabling the closure to be tightened onto the neck and effect a seal adequate to avoid excessive leakage of the contents out of the bottle through an interface between the skirt and neck, interior surfaces of the bottle and closure including the areas of the bottle and closure forming the respective threads being devoid of concave surface areas with a radius smaller than about ⅛″ at corners forming an angle of less than 135°.

2. A combination as set forth in claim 1, wherein the bottle is semi-rigid and capable of being resiliently squeezed for dispensing its contents.

3. A combination as set forth in claim 1, wherein said closure is formed of a material and of a construction substantially stiffer than the bottle whereby the closure is effective in maintaining the bottle neck shape when a main body area of the bottle is squeezed to dispense material from the bottle.

4. A combination as set forth in claim 1, wherein said closure has at least one dispensing hole.

5. A combination as set forth in claim 4, wherein said dispensing hole is formed at a stepped nipple having a rounded step profile to enable the user to select and cut a desired opening.

6. A combination as set forth in claim 1, wherein said bottle has a cylindrical body.

7. A combination as set forth in claim 1, wherein said closure has multiple outlets.

8. A combination as set forth in claim 7, wherein said multiple outlets are arranged on a diametral line across the closure, the closure being circular.

9. In combination, a bottle and top, the bottle and top being molded of a food grade thermoplastic material, the bottle having wall portions forming a main body and a circular neck, the main body and neck having inside surfaces, the top having wall portions forming an end wall and a skirt, the end wall and skirt having inside surfaces, the neck and skirt having mating screw threads permitting the top to be screwed onto the neck, the top and bottle neck having mating surface areas enabling the top to close the bottle at the mating surface areas with a seal to be essentially liquid tight to avoid leakage of contents of the bottle expected to be stored in the bottle, the inside surfaces of the bottle and cap at any inside corner less than 135° and within a zone sealed by said mating surface areas having a radius of at least ⅛″.

10. The bottle and top combination as set forth in claim 9, wherein said bottle is circular and of a size to be grasped in the hand of the average adult.

11. A refillable container suitable for use with edible liquids and other flowable materials, the container comprising a bottle and a mating screw-on top, the bottle having an externally threaded circular upstanding neck and the top having a circular internally threaded depending skirt, the threads of the neck and skirt each extending circumferentially at least one turn about a respective periphery of the neck and skirt, the neck and skirt being of generally uniform respective wall thickness, a root to crest dimension of the threads of both the neck and skirt being greater than the respective wall thickness of the neck and skirt, the threads of the skirt following the profile of the neck threads, the neck threads being proportioned such that concave surface radii of less than ⅛″ are avoided on an interior of the neck, the threads of the neck and skirt resulting in corrugations of the respective walls of the neck and skirt enabling a lip on a mouth of the bottle to more readily conform to an end wall of the top and effectuate a seal therebetween when the top is screw tightened onto the bottle.

12. A method of reducing the risk that a container in the form of a bottle and screw-on cap, which are sold with the bottle empty for use in food service and like commercial activities where the bottle is filled with a food product at the site where it is consumed, used to dispense food product through the cap, and is repeatedly refilled with food product, is not properly cleaned before refilling comprising the steps of blow molding the bottle with an in-turned lip at its mouth for a liquid tight seal with the cap, and molding the cap with respective geometries in which all surface areas within the bottle and cap that are contacted by food product contained in the bottle by the cap are devoid of an inside corner less than ⅛″ in radius whereby the container and cap are rendered easy to clean by scrubbing, flushing, rinsing or the like.

13. A method as set forth in claim 12, wherein the bottle is molded with an external helical screw thread that is reflected in its interior as a groove with a radius of at least ⅛″.

14. A reusable container especially suited for the food service industry comprising a molded bottle and dispensing cap, the bottle having a neck portion with external threads and the cap having mating threads enabling the cap to be screwed onto the bottle, an end wall of the cap being engageable with a lip of a mouth of the bottle to form a liquid tight seal therebetween when the cap is tightened onto the bottle, the threads of the bottle having a radial root to crest height that is about 3 or more times the thickness of a wall area of the neck on which said threads are formed, whereby said bottle is axially compliant in the area of the neck such that its sealing effectiveness with the cap is improved, the bottle and cap having interior surfaces exposed to the contents in the bottle when the cap is sealed on the bottle, said interior surfaces including the inside of the threaded neck area being devoid of inside corners with a radius of less than ⅛″.

15. A reusable container as set forth in claim 14, wherein said bottle is a blow molded product having an in-turned lip at its mouth.

16. A reusable container as set forth in claim 14, wherein said bottle is molded of a relatively flexible semi-rigid material enabling it to be conveniently used as a squeeze bottle to dispense its contents through said cap.
